(19) United States
(12) Patent Application Publication
(10) Pub. No.: US 2019/0345505 A1
(43) Pub. Date: Nov. 14, 2019

(54) METHODS FOR REGULATING EXTRACTABLE PROANTHOCYANIDINS (PAS) IN PLANTS BY AFFECTING LEUCOANTHOCYANIDIN REDUCTASE (LAR)

(71) Applicant: University of North Texas, Denton, TX (US)

(72) Inventors: Richard A. Dixon, Sulphur, OK (US); Chenggang Liu, Denton, TX (US)

(73) Assignee: University of North Texas, Denton, TX (US)

(21) Appl. No.: 16/325,432

(22) PCT Filed: Aug. 15, 2017
(86) PCT No.: PCT/US2017/046929

§ 371 Date: Feb. 14, 2019

Related U.S. Application Data
(60) Provisional application No. 62/375,756, filed on Aug. 16, 2016.

(51) Int. Cl. C12N 15/82

(52) U.S. Cl. CPC C12N 15/8243; C12N 15/8216

(57) ABSTRACT

Adjustments to the amount of soluble and insoluble proanthocyanidins (PAs) in plants can be accomplished through regulation of leucoanthocyanidin reductase (LAR) functionality. Reducing LAR functionality increases epicatechin polymerization, leading to greater amounts of insoluble PAs and effects on astringency and other characteristics.

This application claims priority to U.S. Provisional Patent Application Ser. No. 62/375,756, entitled “Methods for Regulating Extractable Proanthocyanidins (PAs) in Plants by Affecting Leucoanthocyanidin Reductase (LAR),” filed Aug. 16, 2016, the entire contents of which are hereby incorporated by reference.

### BACKGROUND

This disclosure pertains to regulating the content of extractable proanthocyanidins (PAs) in plants.

Proanthocyanidins (PAs) are widely occurring plant-derived oligomers or polymers of flavan-3-ols, predominantly (+)-catechin and (−)-epicatechin, which contribute health benefits for humans, nutritional benefits for livestock, and are an important sink for carbon sequestration. Proanthocyanidins (PAs) are the second most abundant plant polyphenolic compounds after lignin. PAs affect taste, mouthfeel and astringency of many fruits, wines and beverages, have been associated with reduced risks of cardiovascular disease, cancer and Alzheimer's disease, and can improve nutrition and prevent pasture bloat in ruminant animals, as well as enhancing soil nitrogen retention.

PAs may be soluble (extractable) or insoluble depending on the degree to which they are polymerized. Increased polymerization leads to insolubility. Soluble, extractable PAs can be extracted into the juice of a plant or its fruit and will therefore be present in products such as fruit juices or wine. Insoluble PAs remain within the solid portion of the plant, typically bound to cell walls or other components, and will not be present in extracted juice. Adjusting the amount of extractable PAs is important because PAs are known to have nutritional benefits, making an increase in the amount of extractable PAs important. However, they are also known to increase the astringency of fruit juices or wine, making the reduction of extractable PAs important for reducing astringency. The mechanism by which PA monomers polymerize is not understood. Thus, there is currently little understanding of how to internally adjust PA polymerization within a plant in order to regulate the amount of extractable versus insoluble PAs that are present.

### SUMMARY

The present disclosure relates generally to adjusting the amount of soluble and insoluble proanthocyanidins (PAs) in plants through regulation of leucoanthocyanidin reductase (LAR).

Chemically, PAs are oligomers and polymers of flavan-3-ols, primarily (−)-epicatechin and (+)-catechin. As shown in FIG. 1, flavan-3-ols are synthesized through the flavonoid pathway, sharing biosynthetic steps as far as leucoanthocyanidin and anthocyanidin. Leucoanthocyanidin can be converted to (+)-catechin by leucoanthocyanidin reductase (LAR) or to anthocyanidin by anthocyanidin synthase (ANS). Anthocyanidin is then converted to (−)-epicatechin by anthocyanidin reductase (ANR), or processed by a UDP-glucosyl transferase (UGT) to anthocyanin. In spite of many years of research, the mechanism of PA polymerization remains to be determined.

The function of ANR has been demonstrated both genetically and biochemically, but LAR function has only been demonstrated by in vitro biochemical assays and heterologous over-expression in planta. Some plants that produce PAs derived exclusively from epicatechin possess LAR genes, and expression of cacao LAR in tobacco produced more epicatechin than catechin, suggesting that LAR possesses additional functionality.

The present disclosure confirms that loss of LAR functionality increases epicatechin polymerization, leading to greater amounts of insoluble PAs. This is demonstrated particularly with regard to Medicago truncatula, a model legume that possesses a single highly expressed/AR gene, but with seed coat PAs composed almost exclusively of epicatechin. Adjusting the regulation of LAR functionality is expected to have the same effects on any plant that expresses LAR, and particularly on plants known to polymerize PAs in a manner that is affected by LAR. These include the economically important grape, cacao, apple, persimmon, tea, and cranberry plants. The plants contain both epicatechin and LAR genes, indicating a similar function for LAR in these plants. Thus embodiments of the present disclosure pertain to a strategy to control astringency in these plants, and others, through silencing of LAR to facilitate insolublization of PAs.

### BRIEF DESCRIPTION OF THE DRAWINGS

FIG. 1 shows a schematic diagram of the biosynthesis of proanthocyanidins (PAs).

FIG. 2A shows a schematic of the LAR gene depicting Tnt1 insertion positions in lar-1 and lar-2.

FIG. 2B shows RT-PCR for detecting full-length LAR transcripts in R108 (wild-type), lar-1 and lar-2.

FIG. 2C shows qRT-PCR for quantification of Lar transcripts in R108 (wild-type), lar-1 and lar-2.

FIG. 3A shows soluble PA content in wild-type and mutant seeds.

FIG. 3B shows insoluble PA content in wild-type and mutant seeds.

FIG. 3C shows ion abundances in crude extracts from lar mutants.

FIG. 3D shows ion abundances in crude extracts from wild-type seeds.

FIG. 3E shows ion abundances in crude extracts from anr mutants.

FIG. 4A shows a HPLC profile of phloroglucinolysis products from lar-1.

FIG. 4B shows a HPLC profile of phloroglucinolysis products from lar-2.

FIG. 4C shows a HPLC profile of phloroglucinolysis products from wild-type R108.

FIG. 4D shows a HPLC profile of phloroglucinolysis products from procyanidin B2.

FIG. 5A shows a schematic of the Medicago ANR gene depicting Tnt1 insertion positions in arn-1 and arn-2.

FIG. 5B shows soluble PAs quantified by the DMACA method with their contents expressed as epicatechin equivalents.

FIG. 5C shows insoluble PAs quantified by the butanol/HCl method with their contents expressed as procyanidin B2 equivalents.

FIG. 6 shows EIC of flavan-3-ols (289.0718±5 ppm) and epicatechin-3′-O-glucoside (451.1244±5 ppm) in 12 DAP seeds of R108 (wild-type), and lar-1, lar-2 and anr-1 mutants.

FIG. 7 shows quantification of LAR and ANR transcript levels in MYB5 and MYB14 over-expressing hairy roots by qRT-PCR.

FIG. 8A shows EIC of epicatechin and catechin in extracts from MYB5 and MYB14 over-expressing Medicago hairy roots treated with recombinant LAR.

FIG. 8B shows EIC of epicatechin and catechin in extracts from MYB5 and MYB14 over-expressing Medicago hairy roots without treatment with recombinant LAR.

FIG. 9 shows extracts from MYB5 over-expressing Medicago hairy roots separated on a Sep-Pak C18 column and eluted sequentially with increasing concentrations of methanol, F10: 10% methanol fraction, F15: 15%, F20: 20%, F25: 25%, F30: 30%, F40: 40%, and F50: 50%.

FIG. 10A shows HPLC chromatogram of epicatechin and catechin indicating the elution times of the endogenous compounds.

FIG. 10B shows UPLC/MS quantification of epicatechin production from fractions F20 and F25 from FIG. 9 pooled and further separated on an analytical C18 column into 32 fractions (from 5 min to 36 min), then incubated with recombinant LAR.

FIG. 11A shows the mass spectrum of the extract fraction of MYB5-overexpressing M. truncatula hairy roots producing epicatechin.

FIG. 11B shows SIM chromatogram of epicatechin-cysteine from M. truncatula.

FIG. 11C shows MS/MS spectrum of epicatechin-cysteine from M. truncatula.

FIG. 11D shows SIM chromatogram of chemically synthesized 4β-(S-cysteinyl)-epicatechin.

FIG. 11E shows MS/MS spectrum of chemically synthesized 4β-(S-cysteinyl)-epicatechin.

FIG. 12 shows a diagram of ions observed in epicatechin-producing fractions of MYB5 over-expressing hairy roots and their breakdown patterns.

FIG. 13A shows SIM chromatogram of epicatechin-glucuronic acid, where X axis is retention time.

FIG. 13B shows MS/MS spectrum of epicatechin-glucuronic acid, indicating the characteristic ions of glucuronide (m/z 175.02493) and epicatechin carbocation (m/z 287.05600).

FIG. 13C shows SIM chromatogram of epicatechin-glucoside-cysteine, where X axis is retention time.

FIG. 13D shows MS/MS spectrum of epicatechin-glucoside-cysteine, indicating characteristic ions of epicatechin carbocation (m/z 287.05621), epicatechin-cysteine (m/z 408.07650) and epicatechin-glucoside carbocation (m/z 449.10886).

FIG. 14A shows EIC of epicatechin (m/z 289.0718±5 ppm) during conversion of 4β-(S-cysteinyl)-epicatechin to epicatechin by recombinant LAR, including reactions analyzed by UPLC/MS in negative mode without NADPH or LAR, with NADP⁺, and with mutated LAR (LAR/K143G) run in parallel as negative controls.

FIG. 14B shows EIC showing that epicatechin-cysteine (m/z 408.0756±5 ppm) accumulates in lar mutant seeds, but is undetectable in anr mutant seeds.

FIG. 14C shows replicated analyses showing that lar mutant seeds accumulate more epicatechin-cysteine than R108 (wild-type) (n=3).

FIG. 15A shows SDS-PAGE gel of purified recombinant mutated LAR (MBP-LAR/K143G) and wild type LAR (MBP-LAR) fused with maltose binding protein (MBP) stained with coomassie blue.

FIG. 15B shows a plot of initial velocity at different cysteinyl-epicatechin concentrations in a kinetic analysis of recombinant LAR with epicatechin-cysteine as a substrate.

FIG. 15C shows kinetic parameters of wild-type recombinant LAR.

FIG. 16 shows EIC of epicatechin-cysteine in MYB5 and MYB14 over-expressing hairy roots.

FIG. 17A shows EIC of procyanidin dimers formed from auto-polymerization after the incubation of 250 μM cysteinyl-epicatechin and 250 μM epicatechin.

FIG. 17B shows EIC of procyanidin dimers formed from auto-polymerzation after the incubation of 500 μM epicatechin alone.

FIG. 18A shows EIC of procyanidin trimers formed from auto-polymerization after incubation of 250 μM cysteinyl-epicatechin and 250 μM epicatechin.

FIG. 18B shows EIC of procyanidin trimers formed from auto-polymerization after incubation of 500 μM epicatechin alone.

FIG. 19A shows EIC of trimers and tetramers formed from auto-polymerization after incubation of epicatechin with cysteinyl-epicatechin (top panel), with EIC of procyanidin C1 from Arabidopsis seed extract used as standard (bottom panel).

FIG. 19B shows EIC of trimers and tetramers formed from auto-polymerization after incubation of epicatechin with cysteinyl-epicatechin (top panel), with EIC of procyanidin tetramer from Arabidopsis seed extract used as standard (bottom panel).

FIG. 20A shows EIC of trimers from incubation of procyanidin B2 with (top panel) or without (middle panel) cysteinyl-epicatechin, with EIC of procyanidin C1 from Arabidopsis seed extract used as standard (bottom panel).

FIG. 20B shows EIC of tetramers from incubation of procyanidin B2 with (top panel) or without (middle panel) cysteinyl-epicatechin for 24 h, with EIC of epicatechin tetramer from Arabidopsis seed extract used as standard (bottom panel).

FIG. 21 shows a schematic of auto-polymerization products from incubation of cysteinyl-epicatechin with stable ¹³C isotope labeled epicatechin.

FIG. 22A shows EIC of light dimers formed between cysteinyl-epicatechin and ¹³C-labeled epicatechin at various concentration (from 0 μM to 1000 μM) of cysteinyl-epicatechin and 250 μM ¹³C-labeled epicatechin.

FIG. 22B shows EIC of heavy dimers formed from condensation of ¹³C-labeled epicatechin.

FIG. 22C shows EIC of trimers formed between cysteinyl-epicatechin and ¹³C-labeled epicatechin at various concentrations of cysteinyl-epicatechin and 250 μM ¹³C-labeled epicatechin.

FIG. 23A shows EIC of light dimers formed between cysteinyl-epicatechin and ¹³C-labeled epicatechin at various concentrations of ¹³C-labeled epicatechin (from 0 μM to 1000 μM) and 250 μM cysteinyl-epicatechin.

FIG. 23B shows EIC of heavy dimers formed from ¹³C-labeled epicatechin alone.

FIG. 23C shows EIC of trimers formed between cysteinyl-epicatechin and ¹³C-labeled epicatechin at various concentration of ¹³C-labeled epicatechin and 250 μM cysteinyl-epicatechin.

FIG. 24A shows quantification of procyanidin B2 (light B2 and heavy B2) and procyanidin C1 from incubation of various concentrations of cysteinyl-epicatechin (Epi-cys) with a fixed concentration of ¹³C-labeled epicatechin (epi, M+3).

FIG. 24B shows quantification of procyanidin B2 (light B2 and heavy B2) and procyanidin C1 from incubation of various concentrations of ¹³C-labeled epicatechin with a fixed concentration of cysteinyl-epicatechin. Light procyanidin B2 represents the polymerization product between cysteinyl-epicatechin and ¹³C-labeled epicatechin (M+3).

FIG. 24C shows a proposed model of LAR function during PA condensation.

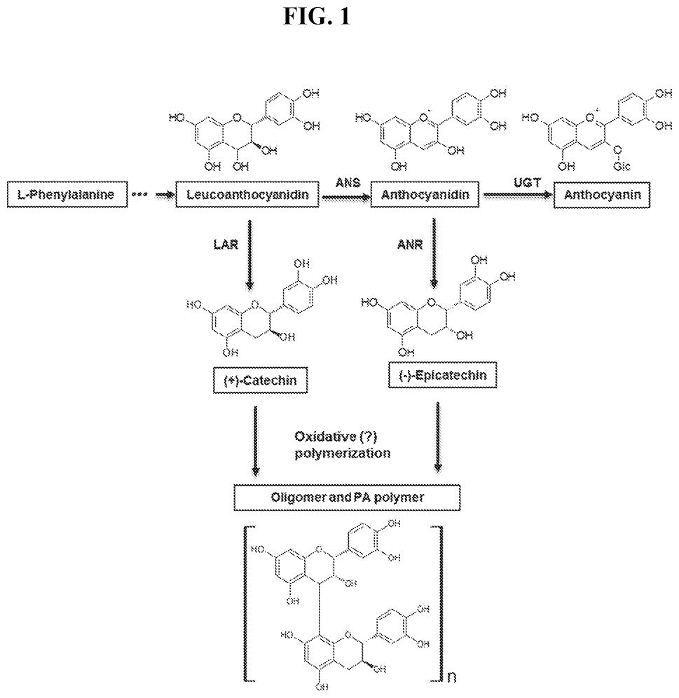

### DETAILED DESCRIPTION OF PREFERRED EMBODIMENTS

Generally, the present disclosure relates to adjusting the amount of proanthocyanidins (PAs) in plants by regulating expression of the gene for leucoanthocyanidin reductase (LAR). 4β-(S-cysteinyl)-epicatechin is demonstrated herein as a conjugate of epicatechin that is a substrate for LAR that provides the 4→8 linked extension units during non-enzymatic PA polymerization. LAR converts 4β-(S-cysteinyl)-epicatechin to epicatechin, the starter unit in PAs, thereby regulating the relative proportions of starter and extension units and consequently the degree of PA oligomerization. By converting 4β-(S-cysteinyl)-epicatechin to epicatechin, LAR removes these extension units necessary for polymerization and thereby inhibits PA oligomerization in the plant. This leads to an increase in soluble PAs and a reduction in insoluble PAs. Loss-of-function of LAR leads to accumulation of 4β-(S-cysteinyl)-epicatechin, increased PA polymerization, increased levels of insoluble PAs, and loss of soluble epicatechin-derived PAs.

In preferred embodiments, the LAR expression can be altered by mutation (such as by transposon insertion). Absent a transposon insertion population in the target plant, LAR expression could also be reduced or eliminated by any method known to those in the art, such as by Crispr CAs9 genome editing, or by RNA interference.

Preferred embodiments include a method for producing a modified plant having increased insoluble proanthocyanidin (PA) content in cells of the plant compared to an unmodified plant of the same species. In additional embodiments, the method includes introducing a mutation into a leucoanthocyanidin reductase (lar) gene in substantially all cells of a plant, wherein the mutation results in reduced or eliminated expression of the leucoanthocyanidin reductase (tar) gene. The modified plant that is produced has reduced or eliminated expression of the leucoanthocyanidin reductase (lar) gene and increased insoluble proanthocyanidin (PA) content in cells of the modified plant.

While preferred embodiments herein are demonstrated particularly with regard to a Medicago truncatula plant, the plant can be a grape, cacao, apple, persimmon, tea or cranberry plant. The modified plant having reduced or eliminated expression of the leucoanthocyanidin reductase (tar) gene and increased insoluble proanthocyanidin (PA) content also has reduced astringency compared to unmodified plants of the same species.

Additional preferred embodiments include a modified plant having increased insoluble proanthocyanidin (PA) content in cells of the plant compared to an unmodified plant of the same species, wherein substantially all cells of the plant comprise a mutation in a leucoanthocyanidin reductase (lar) gene found in the cells of the plant, and wherein the mutation results in reduced or eliminated expression of the leucoanthocyanidin reductase (lar) gene. Further preferred embodiments may include a seed of the modified plant. The modified plant, which may be a Medicago truncatula plant, or a grape, cacao, apple, persimmon, tea or cranberry plant, has reduced astringency compared to unmodified plants of the same species.

### Example 1

Wild-type plants in these examples refer to Medicago truncatula ecotype R108. lar and anr mutants were isolated by screening a tobacco Tnt1 transposon mutagenized Medicago R108 population as described by Tadege et al (13). lar-1 (NF9870), lar-2 (NF18997), arn-1 (NF9161) and arn-2 (NF18737) were obtained from The Noble Foundation, Ardmore, Okla. Seeds were scarified with concentrated sulfuric acid for 10 min, then washed with a large amount of water five times to remove sulfuric acid. Scarified seeds were sterilized with 10% bleach for 10 min and then rinsed five times with sterile water. Sterilized seeds were vernalized at 4° C. for 4 days on moist, sterile filter paper. Vernalized seeds were germinated on filter paper for 5 days before transfer to soil in pots. The plants were grown in a growth chamber set at 16 h/8 h day/night cycle, 22° C.

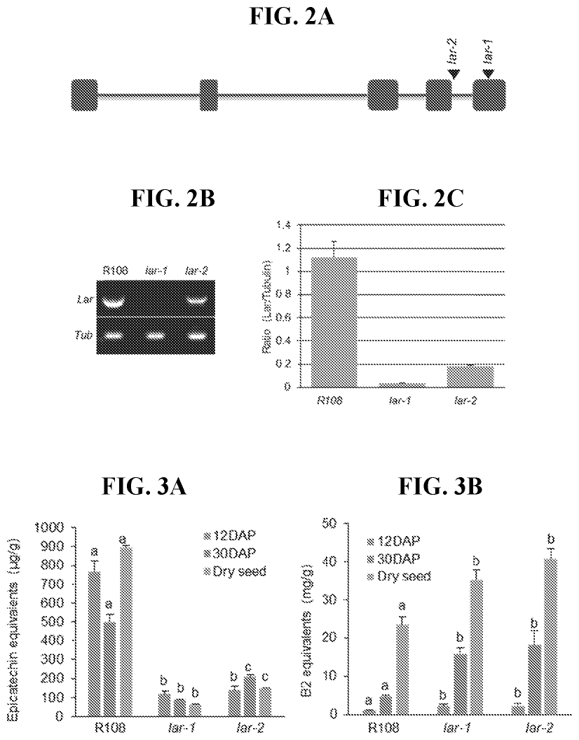

To understand the function of LAR, a Tnt1 transposon mutagenized population of Medicago 13 was screened and two independent mutant alleles were obtained, lar-1 and lar-2, harboring Tnt1 insertions in the last exon and intron of the LAR gene, respectively. FIG. 2A shows a schematic of the LAR gene depicting Tnt1 insertion positions in lar-1 and lar-2. Boxes represent exons while lines represent introns. Tnt1 insertional mutants were confirmed by PCR with gene specific primers and a Tnt1 transposon specific primer. The following primers were used. For lar-1; Tnt1-F, ACAGTGCTACCTCCTCTGGATG (SEQ ID NO:1) and LAR-R, TCAACAGGAAGCTGTGATTGGCACT (SEQ ID NO:2). For lar-2; Tnt1-R, TGTAGCACCGAGATACGGTAATTAACAAGA (SEQ ID NO:3) and LAR-R. For arn-1, Tnt1-F and ANR-R, TCACTTGATCCCCTGAGTCTTCAAATACT (SEQ ID NO:4). For arn-2; ANRGT-F, CCGTGTATGAGTCTATGCTTCATAGCTGT (SEQ ID NO:5) and Tnt1-R.

FIG. 2B shows RT-PCR for detecting full-length LAR transcripts in R108 (wild-type), lar-1 and lar-2 where the PCR was run for 35 cycles. RNA was isolated from 12 DAP seeds dissected from pods, using a Qiagen RNAeasy kit (Qiagen) according to the manufacturer's instructions. RNA was treated with DNase I to remove trace amounts of DNA contamination. One μg of total RNA was used for reverse transcription with SuperScript® III Reverse Transcriptase (Thermo Fisher). qPCR was performed using an ABI QuantStudio™ 6 Flex Real-Time PCR System. For regular RT-PCR, primers LAR-F: CACCATGGCACCATCATCATCAC (SEQ ID NO:6) and LAR-R: TCAACAGGAAGCTGTGATTGGCACT (SEQ ID NO:7) were used for amplification of full-length LAR transcripts. The primers Tub-F, TTTGCTCCTCTTACATCCCGTG (SEQ ID NO:8) and Tub-R, GCCATGGAGAGACCTCTAGG (SEQ ID NO:9), were used for tubulin gene amplification. For qRT-PCR, LAR transcripts were quantified using the primers LARqper-F, CCGTTGGATCAATTGCACATC (SEQ ID NO:10), and LARqper-R, GTAACAGTTGGTAGAGGGTCG (SEQ ID NO:11), and tubulin transcripts were quantified using the primers TUBqPCR-F, TTTGCTCCTCTTACATCCCGTG (SEQ ID NO:12) and TUBqPCR-R, GCAGCACACATCATGTTTTTGG (SEQ ID NO:13).

As seen in FIGS. 2B and 2C, full-length transcripts of LAR were undetectable in homozygous lar-1, whereas low levels were detected in homozygous lar-2. FIG. 2C shows qRT-PCR for quantification of LAR transcripts in R108 (wild-type), lar-1 and lar-2. Transcript levels were averages of 3 independent biological samples. Error bars are standard deviations.

The full length LAR cDNA was cloned into pMal-c5x vector (New England Biolab) at the XmnI and BamHI sites. Mutations, which convert the lysine 143 codon to a glycine codon, were introduced into LAR cDNA by over-lapping PCR. The expression constructs were transformed into E. coli strain Rosetta™ 2(DE3)pLysS (EMD Millipore) competent cells. Transformed bacteria were grown in LB medium supplemented with 0.2% glucose to OD 600 of 0.5, and IPTG was added at 0.3 mM to induce protein expression. Bacteria were harvested after 4 h induction. LAR proteins were purified with amylose resin (NEB, E8021) following the manufacturer's protocol. Briefly, bacteria were lysed by sonication at 4° C. in extraction buffer (20 mM Tris pH 7.0, 200 mM NaCl, 1 mM DTT, 1 mM PMSF). The bacterial lysates were centrifuged at 12,000 g for 15 min at 4° C. The supernatants were loaded on amylose resin which was washed with wash buffer (extraction buffer minus PMSF). Finally, proteins were eluted by elution buffer (20 mM Tris pH 7.0, 200 mM NaCl, 1 mM DTT, 10 mM maltose). Purified proteins were concentrated with an Amicon® Ultra-4 Centrifugal Filter (Millipore) and aliquoted to store at −80° C.

PA content was measured as described by Pang et al. with minor modifications. Briefly, about 50 mg of fresh seeds dissected from the indicated developmental stages, or dry seeds, were ground into powder in liquid nitrogen. The powder was extracted with 1 mL of proanthocyanidin extraction solvent (PES, 70% acetone with 0.5% acetic acid) by sonicating in a water bath for 30 min at room temperature. The resulting slurry was centrifuged at 3000 g for 5 min and supernatants were collected. The pellets were re-extracted twice, all supernatants were pooled, and pellets were saved for analysis of insoluble PAs. Equal volumes of chloroform was added to pooled supernatants and the mixtures vortexed for 30 s, centrifuged at 3000 g for 5 min, and the supernatant further extracted twice with chloroform and twice with hexane. The resulting aqueous phase (soluble PA fraction) was lyophilized and re-dissolved in 50% methanol. PAs in the soluble fraction were quantified by the DMACA method. Five μL of soluble PA fraction were mixed with 200 μL of 0.2% DMACA in methanol/HCl 1:1, and the OD at 640 nm was measured after 5 min. Epicatechin was used as standard.

Insoluble PA content was determined by the butanol/HCl method. The pellet after extraction with PES was lyophilized, 1 mL of butanol/HCl (95:5) was added, and the mixture was sonicated for 1 h to re-suspend the pellet, followed by heating at 95° C. for 1 h. The mixture was then allowed to cool, centrifuged at 12,000 g for 10 min, and the OD at 530 nm was measured. Procyanidin B2 was used as standard and processed in parallel with experimental samples.

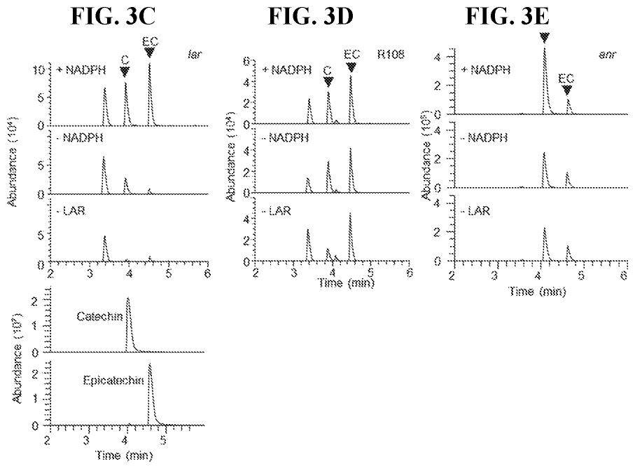

FIG. 3 shows the characterization of lar and anr mutants in M. truncatula. Soluble PA (FIG. 3A) and insoluble PA (FIG. 3B) contents were measured in young seeds (12 DAP), mature wet seeds (30 DAP) and dry seeds of R108 (wild type) and lar mutants. Soluble PA contents were measured by the DMACA method and expressed as epicatechin equivalents. Insoluble PA contents were measured by the butanol/HCl method and expressed as procyanidin B2 equivalents. All measurements were the average of three independent biological replicates. Error bars show standard deviations. Student t tests were used to check statistical significances among each group of measurements (p<0.05). Results shown in FIG. 3C-3E demonstrate that LAR generates epicatechin. Crude extracts from lar (FIG. 3C), R108 (FIG. 3D) and anr (FIG. 3E) were treated with recombinant LAR enzyme. Reactions without NADPH or LAR enzyme were run as negative controls. Reactions were analyzed by UPLC/MS in negative mode, and extracted ion chromatograms (EICs) of catechin (C) and epicatechin (EC), (m/z 289.0718±5 ppm), are presented. It should be noted that the ion abundances in (FIG. 3E) are not comparable with those of FIGS. 3C and 3D, due to the different batches of sample preparation and MS runs.

As shown in FIG. 3A, there was nearly 10-fold less extractable PA present in dry, young (12 DAP), or mature wet (30 DAP) seeds of lar-1, and around 5-fold less in lar-2. In contrast to soluble PAs, insoluble PA levels were higher in lar mutant seeds compared to wild type, shown in FIG. 3B. lar-2 seeds were larger than lar-1 seeds, shown in FIG. 3F suggesting that other mutations may affect seed development in lar-2, which could alter the ratio of PA containing cells in the seeds.

### Example 2

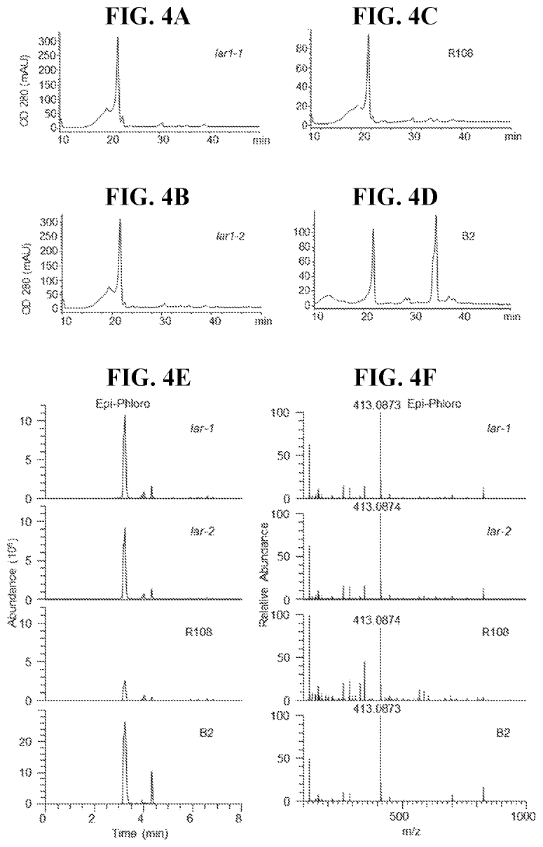

To confirm the nature of the insoluble PAs in lar mutants, the extracted PAs were subjected to phloroglucinolysis followed by HPLC and UPLC/MS analyses. FIG. 4 shows an analysis of the nature of the insoluble PA fraction obtained from the R108 (wild type) and lar mutants described in Example 1 above. Insoluble PAs from R108 and lar mutants were hydrolyzed in the presence of phloroglucinol-HCl. Phloroglucinolysis of insoluble PA fractions was performed by a modification of the procedure described by Pang et al. for soluble PAs. Briefly, the pellet after PES extraction was lyophilized and 200 μL phloroglucinolysis solution (50 mg/mL phloroglucinol, 10 mg/mL ascorbate acid, 0.1N HCl in methanol) was added. The pellet was re-suspended by vortexing and incubated at 50° C. for 20 min. The reaction was terminated by addition of an equal volume of 0.2 M sodium acetate, followed by centrifugation at 12,000 g for 10 min. The supernatant was loaded onto a Sep-Pak C18 column (Waters, Sep-Pak Plus Light) to remove salts and eluted with 50% methanol. The eluted fraction was dried in a speed vacuum centrifuge, dissolved in water, and analyzed by HPLC and UPLC/MS.

HPLC analysis was carried out on Agilent HP1100 system equipped with diode array detector. A 250 mm×4.6 mm, 5 μm, C18 column was used for separation (Varian Metasil 5 Basic). The elution procedure was as follows: Solvent A (water), Solvent B (methanol), flow rate 1 mL/Min. Gradient: 0-5 min, 5% B; 5-20 min, 5%-25% B; 20-40 min, 25%-50% B; 40-50 min, 50%-100% B; 50-60 min, 100% B. Elution profile was monitored at OD 280 nm.

UPLC/MS was carried out on Accela 1250 (Thermo Fisher) system equipped with an Exactive™ Orbitrap mass spectrometer (Thermo Fisher). A 100 mm×2.1 mm, 1.9 μm, C18 column (HypersilGold, Thermo Fisher) was used for separation. The elution procedure was as follows: Solvent A, 0.1% formic acid in water; Solvent B, 0.1% formic acid in methanol; Flow rate, 0.4 mL/min; gradient, 0-1 min, 5% B; 1-2 min, 5%-10% B; 2-13 min, 10%-50% B; 13-14 min, 50%-95% B; 15-15 min, 95% B. The mass spectrometer was set to scan from m/z 100-2000 in negative mode. Selected ion mass spectrometry (SIM) MS/MS analysis was performed with an Orbitrap Velos Pro™ (Thermo Fisher) mass spectrometer coupled with a UPLC system.

HPLC profiles of phloroglucinolysis products are shown in FIG. 4A for lar-1, in FIG. 4B from lar-2, in FIG. 4C for wild-type R108, and in FIG. 4D for procyanidin B2. FIG. 4E shows EIC of released epicatechin phloroglucinol (Epi-phloro, m/z 413.0873±5 ppm). FIG. 4F shows mass spectra for the same biological materials analyzed in FIGS. 4A-4D. Epicatechin-phloroglucinol released from procyanidin B2 was analyzed for comparison. All ions were detected in negative mode. Different HPLC systems were used prior to UV detection and mass spectrometry. More epicatechin-phloroglucinol conjugate (representing epicatechin extension units, identity confirmed by mass spectrometry using procyanidin B2 [epicatechin dimer] as standard), were released from the insoluble PA fraction of lar mutants than from wild-type plants. Based on these observations, loss of function of LAR increases epicatechin polymerization.

### Example 3

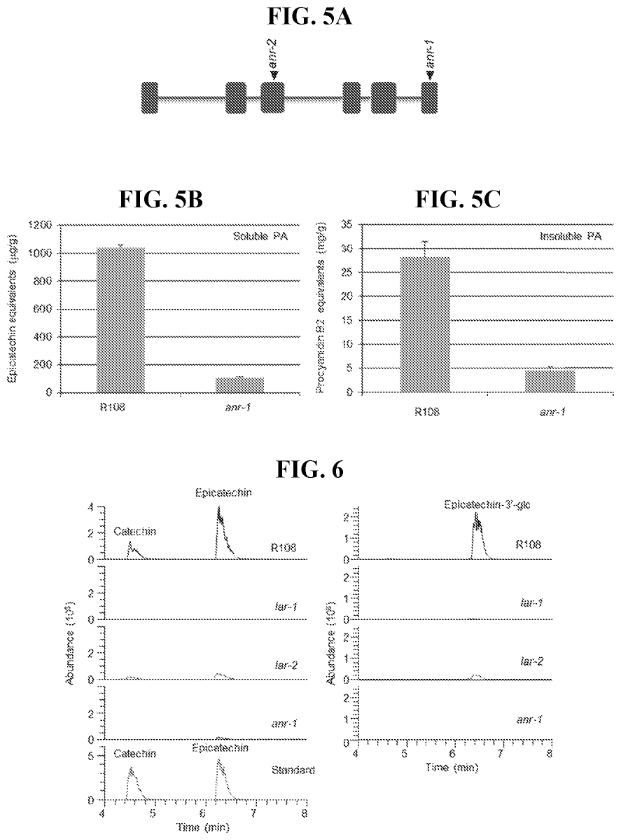

To clarify the relative positions of LAR and ANR in relation to epicatechin in PA biosynthesis, Tnt1 insertion mutants were also examined in ANR. Two mutants were isolated with Tnt1 insertions in the third and sixth exons, respectively. FIG. 5A shows a schematic of the Medicago ANR gene depicting Tnt1 insertion positions in arn-1 and arn-2. Boxes represent exons, while lines represent introns. FIG. 5B shows soluble PAs quantified by the DMACA method with their contents expressed as epicatechin equivalents. FIG. 5C shows insoluble PAs quantified by the butanol/HCl method with their contents expressed as procyanidin B2 equivalents. Loss of function of anr gave large reductions in both soluble and insoluble PAs compared to wild type, consistent with the proposed function of ANR in generating (−)-epicatechin. The low amounts of epicatechin and its 3′-O-glucoside in the anr mutant (seen in FIG. 3E and FIG. 6) could result from non-enzymatic epimerization of catechin to epicatechin, or enzymatic conversion of catechin to epicatechin via anthocyanidin. Treating extracts from seeds of the anr mutant with LAR gave more catechin, but not epicatechin (see FIG. 3E), indicating that anr mutant seeds contain leucocyandin for catechin production, but no substrate for epicatechin production.

The seed color of lar mutants was indistinguishable from wild-type, consistent with PA biosynthesis not being disrupted, whereas the seeds of anr mutants were dark-red resulting from redirected metabolic flow from anthocyanidin to anthocyanin. The lar/anr double mutant displayed the same seed color as the anr mutant, indicating that lar is hypostatic to anr and that ANR functions upstream of LAR. Together, the results indicated that the new substrate of LAR was synthesized after epicatechin and was therefore likely some conjugate of epicatechin.

### Example 4

Because Medicago seed PAs contain almost exclusively epicatechin, it was determined that the lar mutants might accumulate a substrate for LAR other than leucocyanidin (which would be converted by LAR to catechin). To confirm this, crude extracts from 12 DAP seeds of lar-1 mutant and wild type plants were prepared. Twelve DAP seeds (about 100 mg) were dissected from pods and ground to powder in liquid nitrogen. One mL of 80% methanol was added to the powder which was then extracted for 16 h at 4° C. The extract was centrifuged at 12,000 g at room temperature for 10 min, and the methanolic supernatant transferred to a new tube and dried under vacuum. The dried extract was dissolved in 200 μL water and centrifuged for 10 min at 12,000 g at room temperature. Fifty μl of the extract was used for each LAR assay. The LAR reaction was set up in 100 μL volume including 50 mM Tris buffer pH 7.0, 50 μM NADPH, 50 μL crude extract, and 20 μg recombinant LAR protein. The reaction was carried out for 1 h at room temperature and terminated by addition of 200 μL it ethyl acetate to extract the reaction products. The ethyl acetate extract was dried under vacuum, re-dissolved in water and analyzed by UPLC/MS.

FIG. 6 shows EIC of flavan-3-ols (289.0718±5 ppm) and epicatechin-3′-O-glucoside (451.1244±5 ppm) in 12 DAP seeds of R108 (wild-type), and the lar-1, lar-2 and arn-1 mutants. All ions were detected in negative mode. Extracts from lar-1 mutant seeds contained almost no epicatechin, catechin or epicatechin-3′-O-glucoside, whereas wild-type seeds contained epicatechin, epicatechin-3′-O-glucoside and trace amount of catechin (FIGS. 3C and 3D, FIG. 6). Treating extracts of lar-1 mutant with recombinant LAR protein resulted in NADPH-dependent increases in both epicatechin (major product) and catechin (FIG. 3C). A slight increase in both epicatechin and catechin in minus NADPH controls can be explained by the observation that recombinant LAR purified from E. coli is frequently associated with NADPH 15. Low amounts of catechin were present in extracts of young seeds of wild-type plants, although in mature seeds no catechin could be detected as extension or starter units in PAs (FIG. 4). Treating extracts from wild-type plants with recombinant LAR resulted in only small increases in catechin and epicatechin. These results indicate that lar mutant seeds contain a previously uncharacterized substrate that is converted by LAR to epicatechin, as well as a second substrate, presumably leucocyanidin, which is converted to catechin. Wild-type seeds contain the presumptive leucocyanidin and smaller amounts of the epicatechin-producing substrate.

### Example 5

To obtain sufficient material for biochemical characterization of the LAR substrate that is converted to epicatechin, the differential activation of PA pathway genes by transcription factors in Medicago hairy roots was examined. Overexpression of the Medicago MYB14 or MYB5 transcription factors induces PA biosynthesis in hairy roots.

About 50 g of MYB5 over-expressing Medicago hairy roots were grown on 0.7% agar plates containing Gamborg's B-5 medium supplemented with 2% sucrose. Hairy roots were ground to powder in liquid nitrogen and extracted with 500 mL 80% methanol for 16 h at 4° C. Tissue debris was filtered out through four layers of Miracloth (EMD Millipore), and methanol in the extract removed by rotary evaporation at 30° C. The resulting aqueous phase was extracted twice with ethyl acetate to remove endogenous catechin and epicatechin, retained, lyophilized, re-dissolved in 5 mL water and loaded on a Sep-Pak C18 column (Waters, Plus Light) pre-equilibrated with 0.1% formic acid. The column was sequentially washed with 0.1% formic acid, and then 10%, 15%, 20%, 25%, 30%, 40%, 50% methanol containing 0.1% formic acid, 2 mL each wash. Each fraction was lyophilized, re-dissolved in 100 μL water and used as substrate in LAR assays. The fractions containing the most LAR substrate as determined by epicatechin formation (20% and 25% methanol) were further separated by HPLC as described above, with fractions collected every min from 5 min to 36 min. Each fraction was lyophilized and re-dissolved in 100 μL water; half was used as substrate for LAR enzyme assay, and the remaining half was analyzed by UPLC/MS.

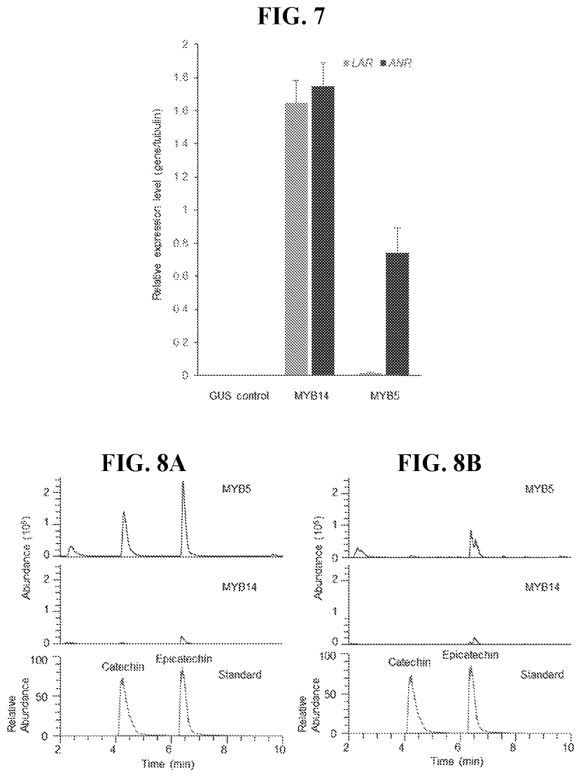

FIG. 7 shows the quantification of LAR and ANR transcript levels in MYB5 and MYB14 over-expressing hairy roots by qRT-PCR. Medicago hairy roots transformed with the same vector harboring the GUS gene was used as vector control. Both ANR and LAR were induced in MYB14 or MYB5 overexpressing hairy roots. However, LAR was induced to much lower level in MYB5 over-expressing hairy roots than in MYB14 over-expressing hairy roots (FIG. 7), suggesting that MYB5 expressing roots might reflect the situation in lar mutant seeds and accumulate the epicatechin-generating substrate of LAR. Indeed, treating extracts from MYB5-, but not MYB14-, over-expressing Medicago hairy roots with recombinant LAR resulted in production of epicatechin, presumably from the same substrate as found in seeds of the lar mutant. FIG. 8 shows EIC of epicatechin and catechin in extracts from MYB5 and MYB14 over-expressing Medicago hairy roots treated with recombinant LAR in (A) Extracts treated with recombinant LAR and (B) Extracts without LAR treatment. All ions were detected in negative mode.

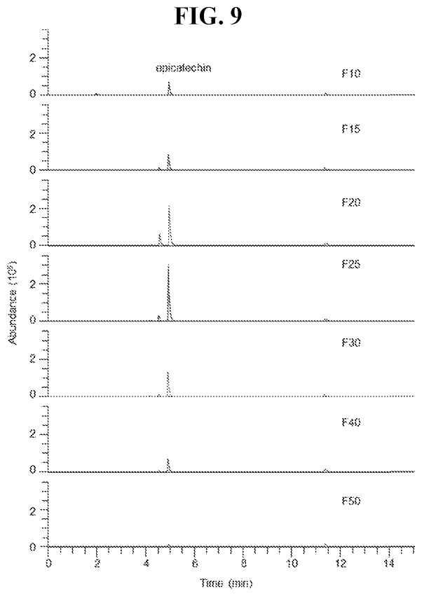

To purify the compound, about 50 g of MYB5-expressing hairy roots was extracted, fractionated on a Sep-Pak SPE C18 column, and then the fractions were treated with recombinant LAR to track the elution of the epicatechin-producing substrate. FIG. 9 shows preliminary fractionation of the substrate of LAR. Extracts from MYB5 over-expressing Medicago hairy roots were separated on a Sep-Pak C18 column and eluted sequentially with increasing concentrations of methanol. F10: 10% methanol fraction, etc. Fractions were then incubated with recombinant LAR. Data show EICs of epicatechin (m/z 289.0718±5 ppm). All ions were detected in negative mode.

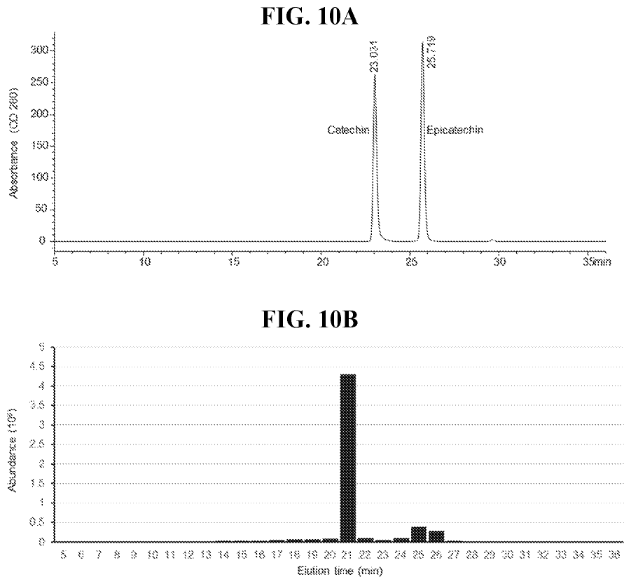

The fractions eluting in 20% and 25% methanol contained the most epicatechin-producing substrate and were further fractionated on an analytical C18 column into 32 fractions. The fraction eluting at 21 min generated the most epicatechin after incubation with recombinant LAR. FIG. 10 shows an analysis of fractions from MYB5-over-expressing Medicago hairy roots for the presence of the substrate of LAR. FIG. 10A shows a HPLC chromatogram of epicatechin and catechin indicating the elution times of the endogenous compounds. FIG. 10B shows Fractions 20 and 25 from the Sep-Pak column (FIG. 9) pooled and further separated on an analytical C18 column into 32 fractions (from 5 min to 36 min), and every fraction was then incubated with recombinant LAR. Epicatechin production was quantified by UPLC/MS analyses with extracted ions. The epicatechin in Fractions 25 and 26 is endogenous free epicatechin.

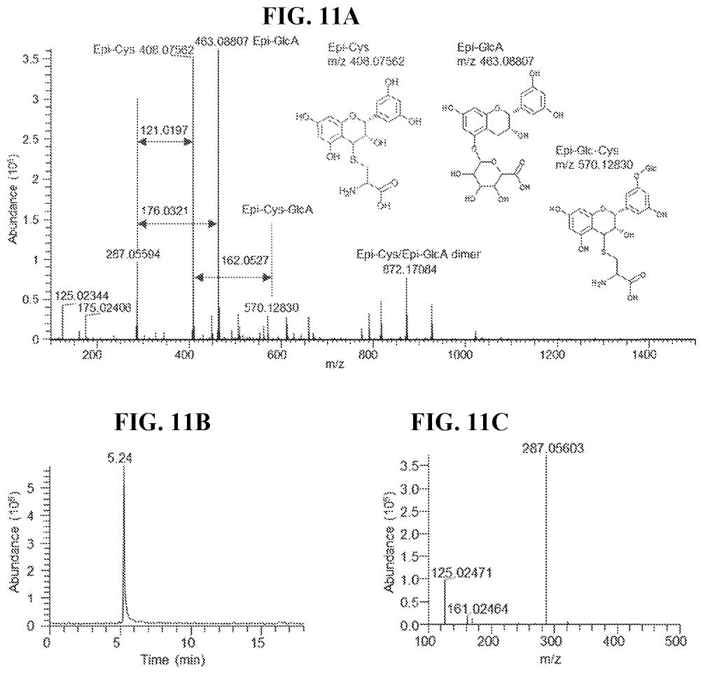

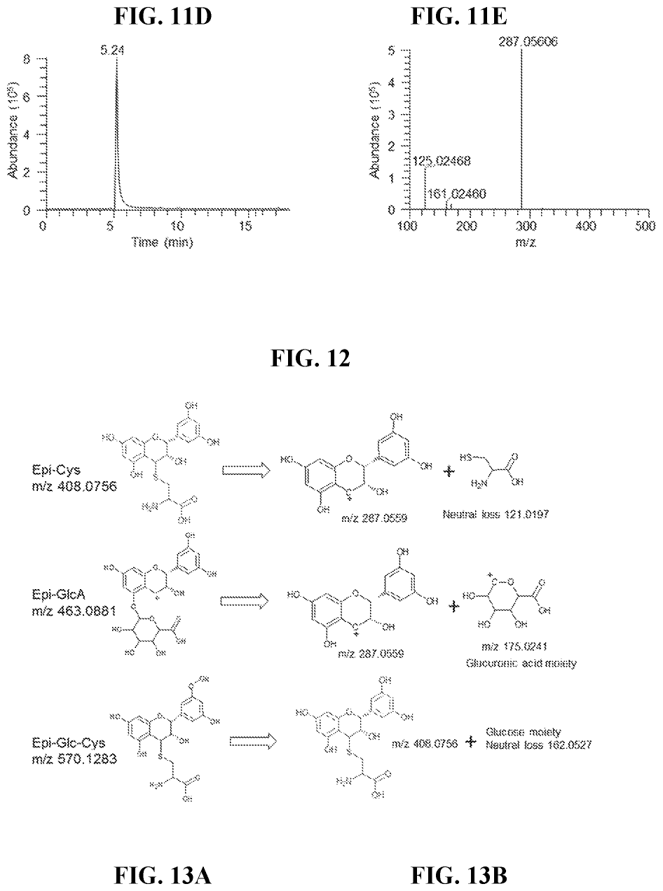

UPLC/accurate mass MS revealed abundant ions of m/z 408.07562, 463.08807 and 287.05594 in this fraction, the latter characteristic of an (epi)catechin carbocation. Extracts of MYB5-overexpressing M. truncatula hairy roots were fractionated by HPLC. The fraction producing epicatechin following incubation with recombinant LAR (fraction 21, FIG. 10) was analyzed by UPLC/MS in negative mode. FIG. 11A shows the mass spectrum of the fraction producing epicatechin. Epi-Cys is the epicatechin-cysteine conjugate; Epi-GlcA is the epicatechin-glucuronic acid conjugate cation. The position of attachment of the glucuronic acid moiety was not determined. A common conjugation position (5-hydroxyl) is shown. Epi-Glc-Cys is the epicatechin-3′-glucoside cysteine conjugate. Glucosylation at the 3′-position is assumed based on previous characterization of epicatechin 3′-O-glucoside. The characteristic neutral losses of cysteine, glucuronic acid and glucose moieties are indicated. FIG. 11B shows SIM chromatogram of epicatechin-cysteine from M. truncatula and FIG. 11C shows its MS/MS spectrum. FIG. 11D shows SIM chromatogram of chemically synthesized 4β-(S-cysteinyl)-epicatechin and FIG. 11E shows its MS/MS spectrum.

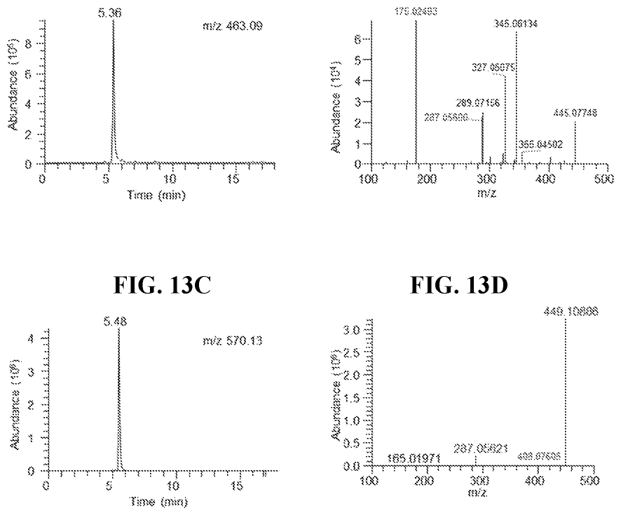

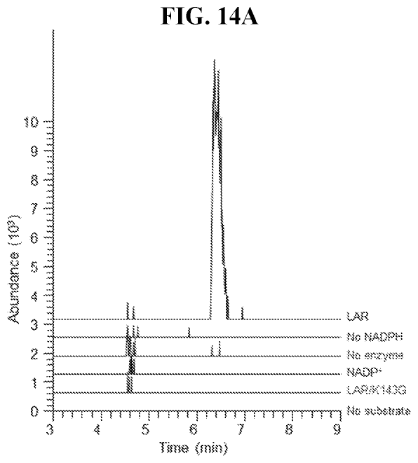

FIG. 12 shows the ions observed in epicatechin-producing fractions of MYB5 over-expressing hairy roots and their breakdown patterns. In addition, an ion with m/z 125.02344, corresponding to the heterocyclic ring fission fragment of epicatechin, was also observed. The neutral loss between 408.07562 and epicatechin carbocation was 121.0197, a characteristic ion loss for cysteine (molecular formula C3H7O2NS). The abundance of m/z 410.07208, the M+2 isotype of m/z 408.07562, was about 4.5% of that of m/z 410.07208, diagnostic for m/z 410.07208 containing sulfur. Therefore m/z 408.07562 was annotated as an epicatechin-cysteine conjugate. Accurate mass analysis of the ion of m/z 463.08807 and its neutral loss breakdown product identified the ion as corresponding to an epicatechin-glucuronide conjugate. Selected ion monitoring (SIM) coupled with MS/MS analysis confirmed that m/z 408.07562 and 463.08807 are parent ions of the epicatechin carbocation. FIG. 13 shows SIM MS/MS analyses of epicatechin-glucuronic acid (m/z 463.09) and epicatechin-glucoside-cysteine (m/z 570.13) conjugates. FIG. 13A shows SIM chromatogram of epicatechin-glucuronic acid, where X axis is retention time. FIG. 13B shows MS/MS spectrum of epicatechin-glucuronic acid, indicating the characteristic ions of glucuronide (m/z 175.02493) and epicatechin carbocation (m/z 287.05600). FIG. 13C shows SIM chromatogram of epicatechin-glucoside-cysteine, where X axis is retention time. FIG. 13D shows MS/MS spectrum of epicatechin-glucoside-cysteine, indicating characteristic ions of epicatechin carbocation (m/z 287.05621), epicatechin-cysteine (m/z 408.07650) and epicatechin-glucoside carbocation (m/z 449.10886). An ion at m/z 570.12830 was annotated as a cysteine conjugate of epicatechin-glucoside, and SIM analyses confirmed that it is the parent ion of 408.07562.

### Example 6

The above analyses indicate that the 21 minute fraction above contains cysteine and glucuronic acid conjugates of epicatechin, as well as a glucoside conjugate of epicatechin cysteine. Of these compounds, epicatechin cysteine was determined to be the best candidate for being a substrate for LAR. The cysteinyl moiety of epicatechin-cysteine is linked at the C4 position of epicatechin. Authentic 4β-(S-cysteinyl)-epicatechin was subsequently synthesized by depolymerization of procyanidin B2 in acidic methanol. More specifically, 4β-(S-Cysteinyl)-epicatechin was synthesized by a modification of the procedure described by Torres et at. Twenty μg procyanidin B2 (Sigma) dissolved in methanol was dried under vacuum, and dissolved in 50 μL lysis solvent containing 18 mg/mL, cysteine base (Sigma), 0.5 N HCl in methanol. The lysis reaction was incubated at 50° C. for 30 min, and the reaction terminated by addition of 200 μL cold water. 4β-(S-Cysteinyl)-epicatechin was purified from the reaction mixture by HPLC using a 250 mm×4.6 mm, 5 μm, C18 column. The fraction containing 4β-(S-cysteinyl)-epicatechin was lyophilized and dissolved in water. To show activity as a substrate for LAR, reaction mixtures containing 50 mM Tris pH 7.0, 50 μM NADPH, 40 μM 4β-(S-cysteinyl)-epicatechin, and 5 μg recombinant LAR protein in a total volume of 50 μL were incubated for 1 h at room temperature and terminated by addition of 200 μL ethyl acetate. The ethyl acetate extract was dried under vacuum, dissolved in 50 μL water and analyzed by UPLC/MS.

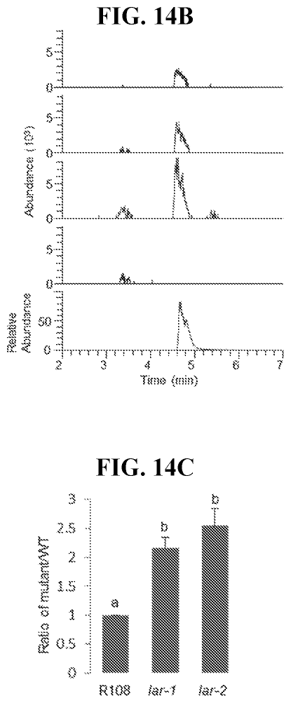

FIG. 14A shows conversion of 4β-(S-cysteinyl)-epicatechin to epicatechin by recombinant LAR. Reactions without NADPH or LAR, with NADP⁺, and with mutated LAR (LAR/K143G) were run in parallel as negative controls. Reactions were analyzed by UPLC/MS in negative mode. The EIC of epicatechin (m/z 289.0718±5 ppm) is presented. Epicatechin-cysteine content was quantified by EIC. FIG. 14 B shows EIC showing that epicatechin-cysteine (m/z 408.0756±5 ppm) accumulates in bar mutant seeds, but is undetectable in anr mutant seeds. FIG. 14C shows replicated analyses showing that bar mutant seeds accumulate more epicatechin-cysteine than R108 (wild-type) (n=3). Student t tests were used to check statistical significance (P<0.05).

The synthesized compound had the same UPLC retention time and MS/MS spectrum as the epicatechin-cysteine conjugate isolated from hairy roots (FIG. 11B-FIG. 11E). Because 4β-(S-cysteinyl)-epicatechin has a sulfur atom at the C4 position, compared to an isovalent oxygen atom in leucocyanidin, it was speculated that LAR might cleave the C—S bond to produce epicatechin, and incubation of authentic 4β-(S-cysteinyl)-epicatechin with LAR generated epicatechin in an NADPH dependent manner (FIG. 14A).

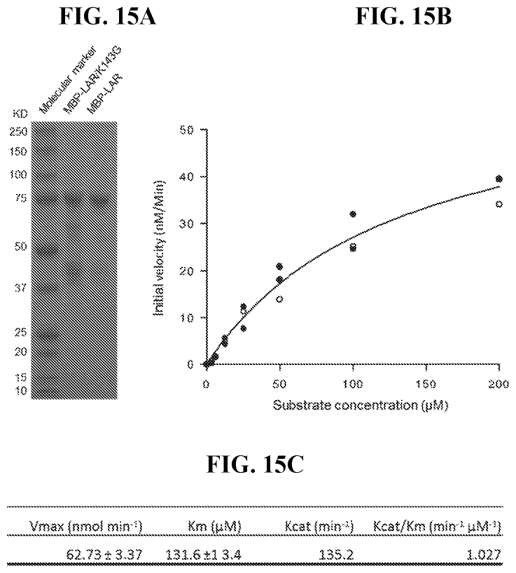

To eliminate the possibility that contamination of recombinant LAR with protein(s) from E. coli might cause the activity, a LAR protein harboring a mutation which converts the conserved lysine 143 to glycine was purified. FIG. 15A shows SDS-PAGE gel of purified recombinant mutated LAR (MBP-LAR/K143G) and wild type LAR (MBP-LAR) fused with maltose binding protein (MBP) stained with coomassie blue. In grape LAR, this conserved lysine (lysine 140) has been shown to be involved in NADPH binding and acts as a general acid catalyst during cleavage of the C4 hydroxyl group of leucocyanidin. Mutating lysine 143 to glycine should therefore abolish the activity of LAR. As shown in FIG. 14A, no activity was observed when incubating authentic 4β-(S-cysteinyl)-epicatechin with mutated LAR, eliminating the possibility that an E. coli protein(s) was responsible for converting 4β-(S-cysteinyl)-epicatechin to epicatechin.

To measure the kinetics of LAR, 1.8 μg of LAR were added to reaction mixtures containing 50 mM Tris pH 7.0, 50 μM NADPH and indicated amounts of 4β-(S-cysteinyl)-epicatechin, Reactions were carried out for 30 min at room temperature to ensure reaction velocities were still increasing in the linear range and terminated by addition of 200 μL ethyl acetate. The ethyl acetate extract was dried under vacuum, dissolved in 50 μL water and analyzed by UPLC/MS. Km and Vmax values were calculated by fitting to the Michaelis-Menten equation with Sigmaplot software. FIG. 15B shows a plot of initial velocity at different cysteinyl-epicatechin concentrations, and FIG. 15C shows kinetic parameters of wild-type recombinant LAR. Kinetic analysis indicated that the Km of LAR towards 4β-(S-cysteinyl)-epicatechin is about 132 μM, and the kcat about 135 Min⁻¹ (FIGS. 15B and 15C). This is significantly higher than the reported Km of LAR from Desmodium uncinatum (6 μM) towards leucocyanidin (Tanner G J, Francki K T, Abrahams S, Watson J M, Larkin P J, Ashton A R (2003) Proanthocyanidin biosynthesis in plants. Purification of legume leucoanthocyanidin reductase and molecular cloning of its cDNA. J Biol Chem 278: 31647-31656). However, this difference is considered of no physiological significance since the two activities of LAR do not compete for the same substrate.

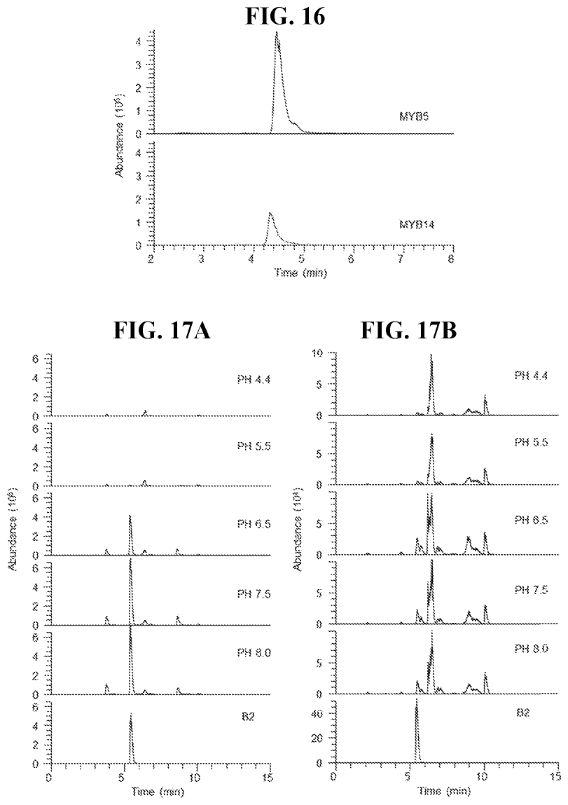

Seeds of the lar mutant accumulated more than twice the level of 4β-(S-cysteinyl)-epicatechin than wild-type plants (FIGS. 14B and 14C), whereas none was detected in the anr mutant, and MYB5 over-expressing hairy roots accumulated more than three times the level of epicatechin-cysteine found in MYB14 over-expressing hairy roots. FIG. 16 shows EIC of epicatechin-cysteine in MYB5 and MYB14 over-expressing hairy roots. DMACA reactivity of epicatechin-cysteine was also measured. Five μL of 1 mM epicatechin or epicatechin-cysteine were added to 200 mL 0.2% DMACA stain solution. OD values of absorbance at 640 nm were measured as: Epicatechin=OD 0.71, Epicatechin-cysteine=OD 0.34, and Blank=OD 0.05. Epicatechin-cysteine reacted with DMACA reagent to produce a less intense blue color than epicatechin, consistent with the weak DMACA staining of lar mutant seeds.

Jiang et al. reported that monomeric flavan-3-ols do not dimerize in auto-polymerization assays, whereas oligomerization occurs with procyanidin B2, either alone or with monomeric flavan-3-ols, suggesting that formation of an epicatechin carbocation to drive dimerization is the crucial step for PA assembly. It was considered that cleavage of the 4β C—S bond of 4β-(S-cysteinyl)-epicatechin would facilitate the formation of epicatechin carbocation, which can attack the C8 position of a terminal epicatechin unit (also known as the starter unit) to initiate oligomerization. To test this, epicatechin was incubated, with or without 4β-(S-cysteinyl)-epicatechin, at various pHs (from 4.4 to 8) and dimerization products were monitored by UPLC/MS. FIG. 17 shows EIC of procyanidin dimers formed from auto-polymerzation between cysteinyl-epicatechin and epicatechin or epicatechin alone at various pH values. FIG. 17A shows results for dimers formed from the incubation of 250 μM cysteinyl-epicatechin and 250 μM epicatechin. FIG. 17B shows dimers formed from the incubation of 500 μM epicatechin alone. B2 refers to procyanidin B2 standard. All ions were detected in negative mode. Note the different scales for the Y axes in FIGS. 17A and 17B. As shown in FIG. 17A, authentic 4→8 linked procyanidin B2 was readily formed above pH 6.5 when epicatechin was incubated with 4β-(S-cysteinyl)-epicatechin. The optimum pH for oligomerization was around 7.5. In contrast, incubation of epicatechin alone produced only trace amount of authentic procyanidin B2 (FIG. 17B) along with a range of different dimers with different elution times, indicating that the oligomerization of epicatchin alone is both random and inefficient (FIG. 17B).

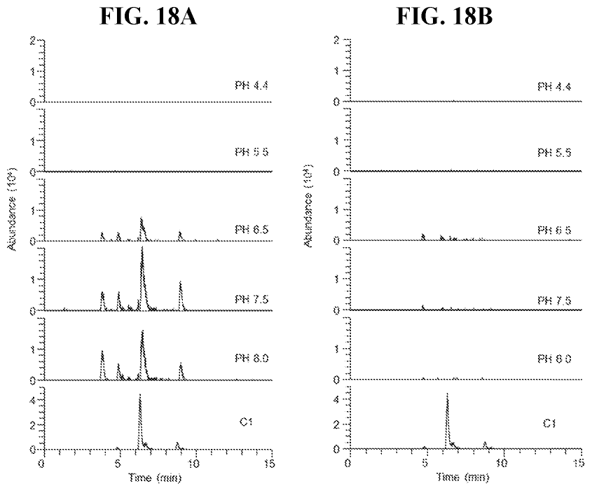

Authentic procyanidin trimers could also be detected when epicatechin was incubated with 4β-(S-cysteinyl)-epicatechin, whereas trimers were not detected on incubation of epicatechin alone. FIG. 18 shows EIC of procyanidin trimers formed from auto-polymerization between cysteinyl-epicatechin and epicatechin or epicatechin alone at various pH values. FIG. 18A shows results for trimers formed from the incubation of 250 cysteinyl-epicatechin and 250 μM epicatechin. FIG. 18B shows results for trimers formed from the incubation of 500 μM epicatechin alone. C1 refers to procyanidin C1 standard from Arabidopsis extracts. All ions were detected in negative mode.

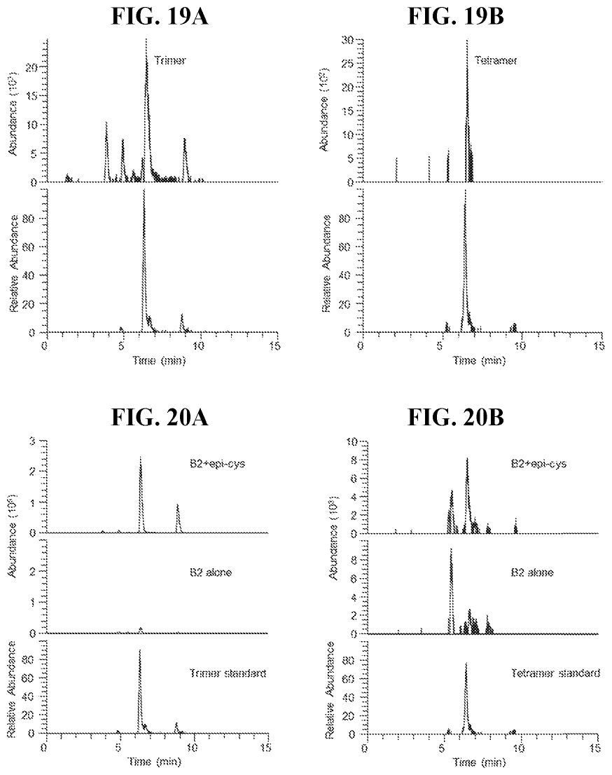

Procyanidin tetramer could be detected after extending the incubation time between 4β-(S-cysteinyl)-epicatechin and epicatechin to 24 h. FIG. 19 shows EIC of trimers and tetramers formed from auto-polymerization between cysteinyl-epicatechin and epicatechin after 24 h incubation. FIG. 19A shows EIC of trimers from incubation of epicatechin with cysteinyl-epicatechin (top panel). EIC of procyanidin C1 from Arabidopsis seed extract was used as standard (bottom panel). FIG. 19B shows EIC of tetramer from incubation of epicatechin with cysteinyl-epicatechin (top panel). EIC of procyanidin tetramer from Arabidopsis seed extract was used as standard (bottom panel).

Procyanidin tetramer could also be detected by incubating procyanidin B2 with 4β-(S-cysteinyl)-epicatechin. FIG. 20 shows EIC of trimer and tetramers formed from auto-polymerization between cysteinyl-epicatechin and procyanidin B2. FIG. 20A shows EIC of trimers from incubation of procyanidin B2 with (top panel) or without (middle panel) cysteinyl-epicatechin. EIC of procyanidin C1 from Arabidopsis seed extract was used as standard (bottom panel). FIG. 20B shows EIC of tetramers from incubation of procyanidin B2 with (top panel) or without (middle panel) cysteinyl-epicatechin for 24 h. EIC of epicatechin tetramer from Arabidopsis seed extract was used as standard (bottom panel).

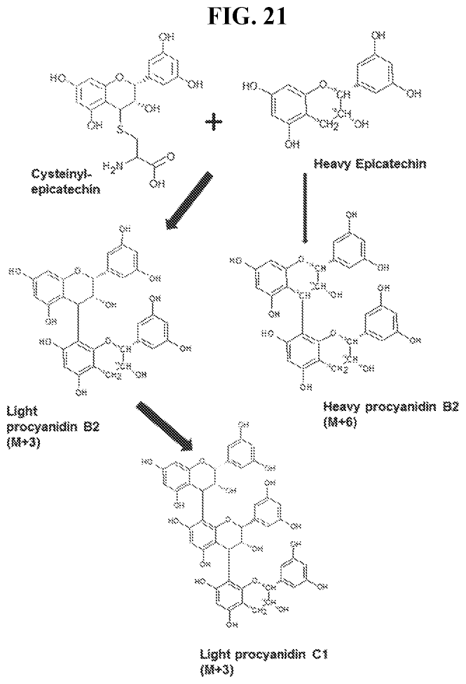

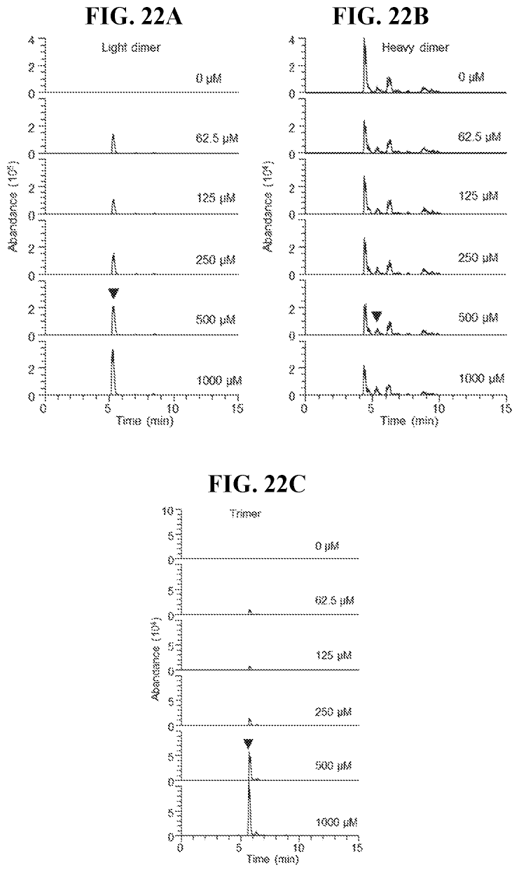

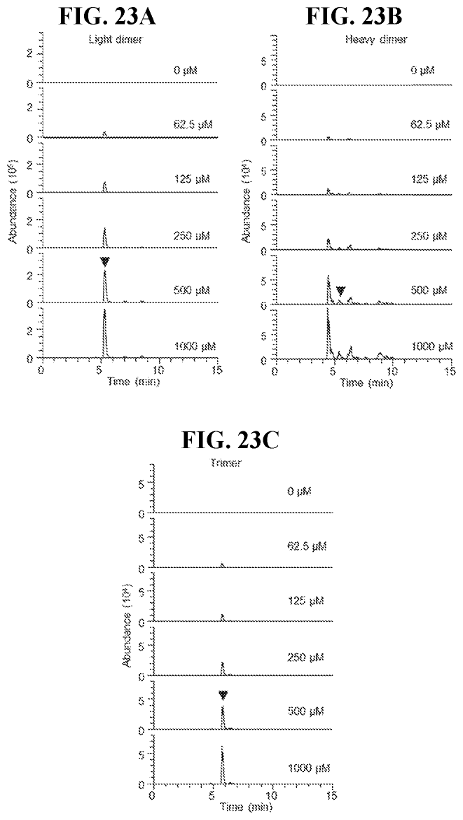

To further demonstrate that 4β-(S-cysteinyl)-epicatechin is the molecule providing the extension unit during procyanidin polymerization, 4β-(S-cysteinyl)-epicatechin was incubated with epicatechin in which the C2, C3 and C4 atoms were labeled with ¹³C. In this way, the dimers or trimers formed between 4β-(S-cysteinyl)-epicatechin and epicatechin could be distinguished from those formed between epicatechin alone by mass spectrometry. Indicated amounts of 4β-(S-cysteinyl)-epicatechin and regular epicatechin or stable 13C isotope labeled epicatechin (Sigma, 719560) were added to a 50 μL reaction volume containing 50 mM potassium phosphate at the indicated pH. Reactions were carried out for 1 h at room temperature unless otherwise indicated and terminated by extraction with 200 μL ethyl acetate. Ethyl acetate extracts were dried under vacuum, dissolved in 50 μL water and analyzed by UPLC/MS. FIG. 21 shows a schematic diagram of auto-polymerization products from incubation of cysteinyl-epicatechin with stable ¹³C isotope labeled epicatechin. FIG. 22 shows EIC of dimers and trimers formed from auto-polymerization between cysteinyl-epicatechin and fixed concentration of ¹³C-labeled epicatechin under different concentrations of cysteinyl-epicatechin. FIG. 22A shows light dimers formed between cysteinyl-epicatechin and ¹³C-labeled epicatechin at various concentrations (from 0 μM to 1000 μM) of cysteinyl-epicatechin and 250 μM ¹³C-labeled epicatechin. FIG. 22B shows heavy dimers formed from condensation of ¹³C-labeled epicatechin. FIG. 22C shows trimers formed between cysteinyl-epicatechin and ¹³C-labeled epicatechin at various concentrations of cysteinyl-epicatechin and 250 μM ¹³C-labeled epicatechin. Note the 10 times difference of Y-axis scale between FIGS. 22A/B and 22C. Triangles indicate the authentic procyanidin B2 and C1 elution times. All ions were detected in negative mode. FIG. 23 shows EIC of dimers and trimers formed from auto-polymerization between cysteinyl-epicatechin and ¹³C-labeled epicatechin at different epicatechin concentrations. FIG. 23A shows light dimers formed between cysteinyl-epicatechin and ¹³C-labeled epicatechin at various concentrations of ¹³C-labeled epicatechin (from 0 μM to 1000 μM) and 250 μM cysteinyl-epicatechin. FIG. 23B shows heavy dimers formed from ¹³C-labeled epicatechin alone. FIG. 23C shows trimers formed between cysteinyl-epicatechin and ¹³C-labeled epicatechin at various concentration of ¹³C-labeled epicatechin and 250 μM cysteinyl-epicatechin. Note the 10 times difference of Y-axis scale between FIGS. 23A and 23B/C. Triangles indicate the authentic procyanidin B2 and C1 elution times. All ions were detected in negative mode

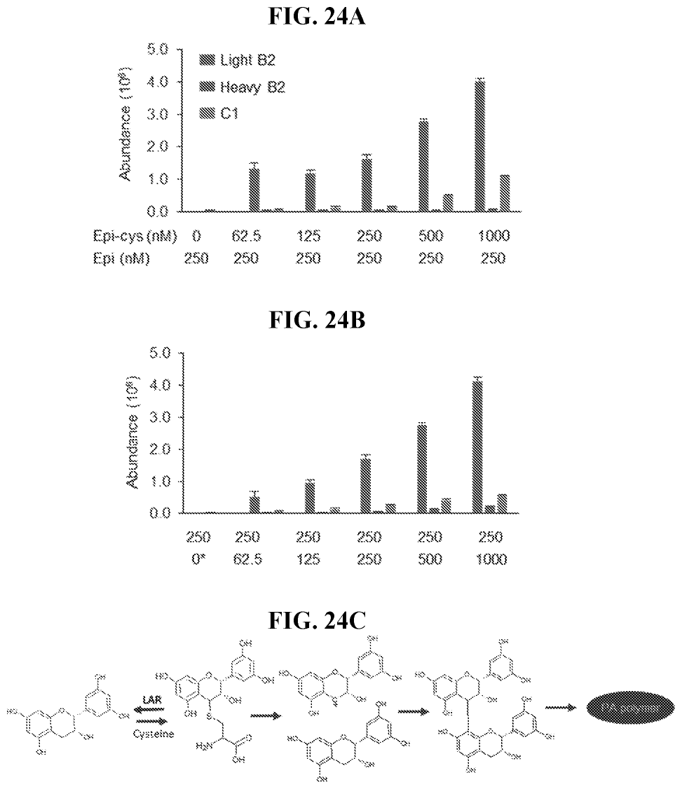

As shown in FIGS. 22 and 23, dimers and trimers were readily detected in this assay. FIG. 24 shows investigation into in vitro auto-condensation between 4β-(S-cysteinyl)-epicatechin and stable isotope-labeled epicatechin. FIG. 24A shows quantification of procyanidin B2 (light B2 and heavy B2) and procyanidin C1 from incubation of various concentrations of cysteinyl-epicatechin (Epi-cys) with a fixed concentration of ¹³C-labeled epicatechin (epi, M+3). FIG. 24B shows quantification of procyanidin B2 (light B2 and heavy B2) and procyanidin C1 from incubation of various concentrations of ¹³C-labeled epicatechin with a fixed concentration of cysteinyl-epicatechin. Light procyanidin B2 represents the polymerization product between cysteinyl-epicatechin and ¹³C-labeled epicatechin (M+3). Heavy procyanidin B2 represents the self polymerization products of ¹³C-labeled epicatechin (M+6). The averages of 3 replicate assays are presented. Error bars are standard deviations. *m/z 577.1348±5 ppm (M) was used to check unlabeled dimers. No unlabeled dimer was detected in this assay. FIG. 24C shows a proposed model of LAR function during PA condensation including epicatechin extension moiety and terminal epicatechin moiety. All ions were detected in negative mode.

The predominant dimer was the procyanidin B2 formed between 4β-(S-cysteinyl)-epicatechin and epicatechin (light B2, M+3) (see FIGS. 24A, 24B, 22A, and 23A). Only trace amounts of dimers formed between two epicatechin molecules (heavy B2, M+6) could be detected (see FIGS. 24A, 24B, 22B, and 23B), and no dimers formed from 4β-(S-cysteinyl)-epicatechin alone (M) could be detected (see FIG. 24B). Only trimers with m/z value M+3 could be detected (see FIGS. 24A, 24B, 22C, and 23C), confirming that the isotope-labeled epicatechin provides only the starter units and 4β-(S-cysteinyl)-epicatechin provides the extension units during procyanidin oligomerization. Increasing the ratio of 4β-(S-cysteinyl)-epicatechin to epicatechin promoted formation of more trimers.

These results indicate that the initiation of PA polymerization occurs between an epicatechin starter unit and an epicatechin carbocation extension unit formed by facile nucleophilic displacement of the Cys leaving group of 4β-(S-cysteinyl)-epicatechin (see FIG. 24C). This reaction can occur non-enzymatically, and may also operate for subsequent extension of the chain to generate a 4→8 linked oligomer/polymer. The origin of the 4β-(S-cysteinyl)-epicatechin could be enzymatic or non-enzymatic, and PA chain length will depend on the relative proportions of starter and extension units. This is determined, at least in Medicago, by the relative activities of the reaction forming 4β-(S-cysteinyl)-epicatechin and of LAR (to convert 4β-(S-cysteinyl)-epicatechin back to epicatechin). Higher concentrations of epicatechin-cysteine lead to a higher degree of polymerization and eventual insolubility of the PAs, as observed in the lar mutants.

Although Medicago possesses a highly expressed LAR gene, encoding an enzyme that catalyzes formation of catechin from leucocyanidin, catechin units are not detectable in mature Medicago seeds and are only present in trace amount in young seeds. This can be explained if the enzyme LDOX has higher affinity for leucocyanidin than has LAR, and channels most of the leucocyanidn to cyanidin which can then form epicatechin through the action of ANR. In this scenario, the major function for LAR in Medicago is the regulation of PA oligomerization through the removal of the activated extension unit 4β-(S-cysteinyl)-epicatechin. This function is supported by the accumulation of 4β-(S-cysteinyl)-epicatechin and a larger proportion of insoluble PAs with near disappearance of soluble PAs including monomers in the lar mutants. Many economically important plants such as grape, cacao, and apple contain both epicatechin and LAR genes, and these results support a similar function for LAR in these plants, as well as a strategy to control astringency through silencing of LAR to facilitate insolublization of PAs.

### REFERENCES CITED

The following documents and publications are hereby incorporated by reference.

### Non-Patent Publications

Gonzalo-Diago, A., Dizy, M. & Fernandez-Zurbano, P. Taste and Mouthfeel Properties of Red Wines Proanthocyanidins and Their Relation to the Chemical Composition. Journal of Agricultural and Food Chemistry 61, 8861-8870, doi: 10.1021/jf401041q (2013).
Lesschaeve, I. & Noble, A. C. Polyphenols: factors influencing their sensory properties and their effects on food and beverage preferences. The American journal of clinical nutrition 81, 330s-335s (2005).
Bagchi, D. et al. Free radicals and grape seed proanthocyanidin extract: importance in human health and disease prevention. Toxicology 148, 187-197, doi: 10.1016/S0300-483X(00)00210-9 (2000).
Cos, P. et al. Proanthocyanidins in health care: current and new trends. Current medicinal chemistry 11, 1345-1359 (2004).
Middleton, E., Jr., Kandaswami, C. & Theoharides, T. C. The effects of plant flavonoids on mammalian cells: implications for inflammation, heart disease, and cancer. Pharmacological reviews 52, 673-751 (2000).
Lees, G. L. Condensed tannins in some forage legumes: their role in the prevention of ruminant pasture bloat. Basic life sciences 59, 915-934 (1992).
Devic, M. et al. The BANYULS gene encodes a DFR-like protein and is a marker of early seed coat development. The Plant journal: for cell and molecular biology 19, 387-398 (1999).
Xie, D. Y., Sharma, S. B., Paiva, N. L., Ferreira, D. & Dixon, R. A. Role of anthocyanidin reductase, encoded by BANYULS in plant flavonoid biosynthesis. Science (New York, N.Y.) 299, 396-399, doi:10.1126/science.1078540 (2003).
Bogs, J. et al. Proanthocyanidin synthesis and expression of genes encoding leucoanthocyanidin reductase and anthocyanidin reductase in developing grape berries and grapevine leaves. Plant physiology 139, 652-663, doi:10.1104/pp. 105.064238 (2005).
Liu, Y., Shi, Z., Maximova, S., Payne, M. J. & Guiltinan, M. J. Proanthocyanidin synthesis in Theobroma cacao: genes encoding anthocyanidin synthase, anthocyanidin reductase, and leucoanthocyanidin reductase. BMC plant biology 13, 202, doi:10.1186/1471-2229-13-202 (2013).
Pang, Y., Peel, G. J., Wright, E., Wang, Z. & Dixon, R. A. Early steps in proanthocyanidin biosynthesis in the model legume Medicago truncatula. Plant physiology 145, 601-615, doi:10.1104/pp. 107.107326 (2007).
Tanner, G. J. et al. Proanthocyanidin biosynthesis in plants. Purification of legume leucoanthocyanidin reductase and molecular cloning of its cDNA. J Biol Chem 278, 31647-31656 (2003).
Tadege, M. et al. Large-scale insertional mutagenesis using the Tnt1 retrotransposon in the model legume Medicago truncatula. The Plant Journal 54, 335-347, doi:10.1111/j.1365-313X.2008.03418.x (2008).
Kiatgrajai, P., Wellons, J. D., Gollob, L. & White, J. D. Kinetics of epimerization of (+)-catechin and its rearrangement to catechinic acid. The Journal of Organic Chemistry 47, 2910-2912, doi:10.1021/jo00136a021 (1982).
Mauge, C. et al. Crystal structure and catalytic mechanism of leucoanthocyanidin reductase from Vitis vinifera. Journal of molecular biology 397, 1079-1091, doi:10.1016/j.jmb.2010.02.002 (2010).
Liu, C., Jun, J. H. & Dixon, R. A. MYB5 and MYB14 Play Pivotal Roles in Seed Coat Polymer Biosynthesis in Medicago truncatula. Plant physiology 165, 1424-1439, doi:10.1104/pp. 114.241877 (2014).
Tones, J. L. et al. Cysteinyl-flavan-3-ol Conjugates from Grape Procyanidins. Antioxidant and Antiproliferative Properties. Bioorganic & Medicinal Chemistry 10, 2497-2509, doi: 10.1016/S0968-0896(02)00127-X (2002).
Tones, J. L., Lozano, C. & Maher, P. Conjugation of catechins with cysteine generates antioxidant compounds with enhanced neuroprotective activity. Phytochemistry 66, 2032-2037, doi: 10.1016/j.phytochem.2004.12.003 (2005).
Jiang, X. et al. Analysis of accumulation patterns and preliminary study on the condensation mechanism of proanthocyanidins in the tea plant [Camellia sinensis]. Scientific Reports 5, 8742, doi:10.1038/srep08742, nature.com/articles/srep08742#supplementary-information (2015).
Liao, L. et al. Molecular characterization of genes encoding leucoanthocyanidin reductase involved in proanthocyanidin biosynthesis in apple. Frontiers in Plant Science 6, 243, doi:10.3389/fpls.2015.00243 (2015).

What is claimed is:

1. A method for producing a modified plant having increased insoluble proanthocyanidin (PA) content in cells of the plant compared to an unmodified plant of the same species, comprising:
reducing or eliminating expression of the leucoanthocyanidin reductase (lar) gene; and
producing a modified plant having reduced or eliminated expression of the leucoanthocyanidin reductase (lar) gene and increased insoluble proanthocyanidin (PA) content in cells of the modified plant.

2. The method of claim 1, wherein the step of reducing or eliminating expression of the leucoanthocyanidin reductase (lar) gene comprises introducing a mutation into a leucoanthocyanidin reductase (lar) gene in substantially all cells of a plant, wherein the mutation results in reduced or eliminated expression of the leucoanthocyanidin reductase (lar) gene.

3. The method of claim 1, wherein the plant is a Medicago truncatula plant.

4. The method of claim 1, wherein the plant is a grape, cacao, apple, persimmon, tea or cranberry plant.

5. The method of claim 1, wherein the modified plant has reduced astringency compared to unmodified plants of the same species.

6. A modified plant having increased insoluble proanthocyanidin (PA) content in cells of the plant compared to an unmodified plant of the same species, wherein substantially all cells of the plant comprise a mutation in a leucoanthocyanidin reductase (lar) gene found in the cells of the plant, and wherein the mutation results in reduced or eliminated expression of the leucoanthocyanidin reductase (lar) gene.

7. A seed of the modified plant of claim 6.

8. The modified plant of claim 6, wherein the modified plant is a Medicago truncatula plant.

9. The modified plant of claim 6, wherein the plant is a grape, cacao, apple, persimmon, tea or cranberry plant.

10. The modified plant of claim 6, wherein the modified plant has reduced astringency compared to unmodified plants of the same species.


SEQUENCE LISTING (13 sequences)

SEQ ID NO: 1 - DNA, 22 bp, Artificial Sequence
  Primer Tnt1-F
acagtgctacctcctctggatg

SEQ ID NO: 2 - DNA, 25 bp, Artificial Sequence
  Primer LAR-R
tcaacaggaagctgtgattggcact

SEQ ID NO: 3 - DNA, 30 bp, Artificial Sequence
  Primer Tnt1-R
tgtagcaccgagatacggtaattaacaaga

SEQ ID NO: 4 - DNA, 29 bp, Artificial Sequence
  Primer ANR-R
tcacttgatcccctgagtcttcaaatact

SEQ ID NO: 5 - DNA, 29 bp, Artificial Sequence
  Primer ANRGT-F
ccgtgtatgagtctatgcttcatagctgt

SEQ ID NO: 6 - DNA, 23 bp, Artificial Sequence
  Primer LAR-F
caccatggcaccatcatcatcac

SEQ ID NO: 7 - DNA, 25 bp, Artificial Sequence
  Primer LAR-R
tcaacaggaagctgtgattggcact

SEQ ID NO: 8 - DNA, 22 bp, Artificial Sequence
  Primer Tub-F
tttgctcctcttacatcccgtg

SEQ ID NO: 9 - DNA, 20 bp, Artificial Sequence
  Primer Tub-R
gccatggagagacctctagg

SEQ ID NO: 10 - DNA, 21 bp, Artificial Sequence
  Primer LARqpcr-F
ccgttggatcaattgcacatc

SEQ ID NO: 11 - DNA, 21 bp, Artificial Sequence
  Primer LARqpcr-R
gtaacagttggtagagggtcg

SEQ ID NO: 12 - DNA, 22 bp, Artificial Sequence
  Primer TUBqPCR-F
tttgctcctcttacatcccgtg

SEQ ID NO: 13 - DNA, 22 bp, Artificial Sequence
  Primer TUBqPCR-R
gcagcacacatcatgtttttgg